(19) United States
(12) Patent Application Publication
(10) Pub. No.: US 2015/0011977 A1
(43) Pub. Date: Jan. 8, 2015

(54) PUNCTURE DEVICE ASSEMBLY

(71) Applicant: TERUMO KABUSHIKI KAISHA, Shibuya-ku (JP)

(72) Inventor: Junko Kuniyasu, Fuji (JP)

(73) Assignee: TERUMO KABUSHIKI KAISHA, Tokyo (JP)

(21) Appl. No.: 14/497,672

(22) Filed: Sep. 26, 2014

Related U.S. Application Data
(63) Continuation of application No. PCT/JP2013/057248, filed on Mar. 14, 2013.

(30) Foreign Application Priority Data
Mar. 28, 2012 (JP) 2012-072976

(51) Int. Cl. A61B 17/34; A61M 39/06; A61M 25/10

(52) U.S. Cl. CPC A61B 17/3415; A61M 25/10; A61M 39/06; A61M 2039/062; A61M 2025/1081
USPC 604/507; 604/164.01; 604/96.01; 604/167.03

(57) ABSTRACT

Provided is a puncture device assembly excellent in operability. Provided is a puncture device assembly which includes an inner needle that can puncture skin, a sheath tube that has a lumen through which the inner needle can be inserted, and a support portion which is provided at a proximal portion of the sheath tube and prevents kink of the sheath tube. The support portion has a cylindrical body which is bendable and can maintain a curved shape in a state where the sheath tube is inserted.

### CROSS-REFERENCES TO RELATED APPLICATIONS

This application is a continuation of International Application No. PCT/JP2013/057248 filed on Mar. 14, 2013, and claims priority to Japanese Application No. 2012-072976 filed on Mar. 28, 2012, the entire content of both of which is incorporated herein by reference.

### TECHNICAL FIELD

The present invention relates to a puncture device assembly.

### BACKGROUND DISCUSSION

Various types of treatments and inspections have been performed using a catheter which is a medical elongated object. Examples of treatment methods include a treatment method of directly administering an agent into an affected area using a catheter; a treatment method of expanding and opening a narrowed section in the lumen in a living body using a catheter in which a balloon expanded by pressure is attached to a distal end thereof; a treatment method of scraping and removing the affected area using a catheter in which a cutter is attached to a distal portion thereof; a treatment method of closing arterial aneurysm, bleeding, or feeding vessel sites with a filling material using a catheter; a treatment method of placing a stent, which maintains an open state of the narrowed section in the lumen in a living body, into the lumen in the living body using a catheter; and a treatment method of aspirating a thrombus that blocks the blood vessels using a catheter.

In general, the catheter is percutaneously inserted into a lesion area such as a blood vessel using a puncture device assembly such as an introducer which has a sheath tube through which the catheter can be inserted (for example, refer to Japanese Patent No. 8-131552).

### SUMMARY

However, there is a problem in that it is difficult to treat the puncture device assembly since the sheath tube of the puncture device assembly has a long length. For example, in a case where the section of the sheath tube positioned outside the body lies down during a procedure, mounting of the catheter is troublesome and there is a problem in operability.

A puncture device assembly is provided which includes: an inner needle that can puncture skin; a sheath tube that has a lumen through which the inner needle can be inserted; and a support portion which is provided at a proximal portion of the sheath tube and prevents kink of the sheath tube, in which the support portion has a cylindrical body which is bendable and can maintain a curved shape in a state where the sheath tube is inserted.

The support portion has a cylindrical body which is bendable and can maintain the curved shape in a state where the sheath tube is inserted. Therefore, it is possible to suppress the lying down of the sheath tube or kink of the sheath itself by maintaining the curved shape of the proximal portion of the sheath tube which is positioned in the vicinity of an insertion point. Accordingly, it is easy to insert a medical elongated object through the lumen of the sheath tube or to attach a medical instrument to the proximal portion of the sheath tube, for example. That is, it is possible to provide a puncture device assembly excellent in operability.

For example, the medical elongated object is a balloon catheter or a stent delivery catheter and the medical instrument is a hemostasis valve.

In a case where the sheath tube also serves as an introducer sheath, that is, in a case where the sheath tube functions as the introducer sheath, the procedure is simplified, which is preferable.

It is preferable that the cylindrical body is formed of a plurality of cylindrical members bendably connected to each other, and that the curved shape of the cylindrical body is maintained based on friction between the adjacent cylindrical members. In this case, it is possible to achieve the structure which is bendable and can maintain the curved shape, with a simple structure. Specific examples of the simple structures include a case where the cylindrical members have a diameter-enlarged distal portion and a truncated cone-shaped base, and the spherical distal portion is rotatably fitted and contacted to the truncated cone-shaped base of the adjacent cylindrical members; and a case where the cylindrical members have the diameter-enlarged distal portion and a diameter-reduced base, and the diameter-enlarged distal portion is rotatably fitted and contacted to the diameter-reduced base of the adjacent cylindrical members.

It is preferable that the puncture device assembly have a hemostasis valve which is integrated with the sheath tube. In this case, the complexity in mounting operation of the hemostasis valve is reduced.

It is preferable that the proximal portion of the sheath tube have a connector that can attach and detach the hemostasis valve. In this case, it is possible to attach and detach the hemostasis valve as necessary, and therefore, it is possible to apply the invention to various procedures.

It is preferable that the distal portion of the sheath tube have a groove generating a reflection echo with respect to ultrasonic waves. In this case, it is possible to specify the position of the distal portion of the sheath tube while visually confirming blood flow from an echo screen in real time even in a circumstance where it is difficult due to less blood flow to perform flashback through which the skin is punctured while confirming the blood flow, and therefore, the puncture device assembly is excellent in operability. It is preferable that the groove be disposed on a surface of a lumen of the sheath tube. In this case, an air layer is interposed between the grooves in a manner of being held by the grooves, and therefore, it is possible to efficiently generate the echo.

A method comprises puncturing a tissue with a cutting edge of a needle, the needle being positioned inside a lumen of a sheath tube wherein the cutting edge of the needle extends beyond a distal end portion of the sheath tube, introducing the distal end portion of the sheath tube to a blood vessel extending inside the tissue; removing the needle from the sheath tube, bending a support portion of the sheath tube to be in a curved shape by applying an external force, the support portion of the sheath tube being provided proximal to the distal end portion of the sheath tube, where the support portion of the sheath tube maintains its curved shape when the external force is removed.

The support portion of the sheath tube includes a plurality of axially adjacent members, and the curved shape of the cylindrical body is maintained based on friction between each pair of axially adjacent cylindrical members. Each of the axially adjacent members has a central axis, where the central axis of the plurality of axially adjacent members are coaxial prior to the external force being applied. Each of the central axis of the plurality of axially adjacent members are eccentric relative to an axially adjacent member when the external force is applied, and each of the central axis of the plurality of axially adjacent members remains eccentric relative to an axially adjacent member when the external force is subsequently removed.

### BRIEF DESCRIPTION OF THE DRAWINGS

FIG. 1 is a side view illustrating a puncture device assembly according to Embodiment 1.

FIG. 2 is a side view illustrating an inner needle shown in FIG. 1.

FIG. 3 is a side view illustrating a sheath tube shown in FIG. 1.

FIG. 4 is a side view illustrating a hemostasis valve attached to a connector shown in FIG. 1.

FIG. 5 is a side view illustrating support portion shown in FIG. 1.

FIG. 6 is a cross-sectional view illustrating the support portion shown in FIG. 1.

FIG. 7 is a side view showing a curved state of the support portion shown in FIG. 1.

FIG. 8 is a side view illustrating puncturing according to a method of using the puncture device assembly according to Embodiment 1.

FIG. 9 is a side view illustrating removal of the inner needle according to the method of using the puncture device assembly according to Embodiment 1.

FIG. 10 is a side view illustrating mounting of the hemostasis valve according to the method of using the puncture device assembly according to Embodiment 1.

FIG. 11 is a side view illustrating insertion of a guide wire according to the method of using the puncture device assembly according to Embodiment 1.

FIG. 12 is a side view illustrating insertion of a balloon catheter according to the method of using the puncture device assembly according to Embodiment 1.

FIG. 13 is a side view illustrating Modification Example 1 according to Embodiment 1.

FIG. 14 is a cross-sectional view illustrating Modification Example 1 according to Embodiment 1.

FIG. 15 is a side view showing a curved state of Modification Example 1 according to Embodiment 1.

FIG. 16 is a cross-sectional view illustrating Modification Example 2 according to Embodiment 1.

FIG. 17 is a cross-sectional view illustrating Modification Example 3 according to Embodiment 1.

FIG. 18 is a side view illustrating a puncture device assembly according to Embodiment 2.

FIG. 19 is a side view illustrating puncturing according to a method of using the puncture device assembly according to Embodiment 2.

FIG. 20 is a side view illustrating removal of an inner needle according to the method of using the puncture device assembly according to Embodiment 2.

FIG. 21 is a side view illustrating insertion of a guide wire according to the method of using the puncture device assembly according to Embodiment 2.

FIG. 22 is a side view illustrating insertion of a balloon catheter according to the method of using the puncture device assembly according to Embodiment 2.

### DETAILED DESCRIPTION

Hereinafter, embodiments of the present invention will be described with reference to the accompanying drawings.

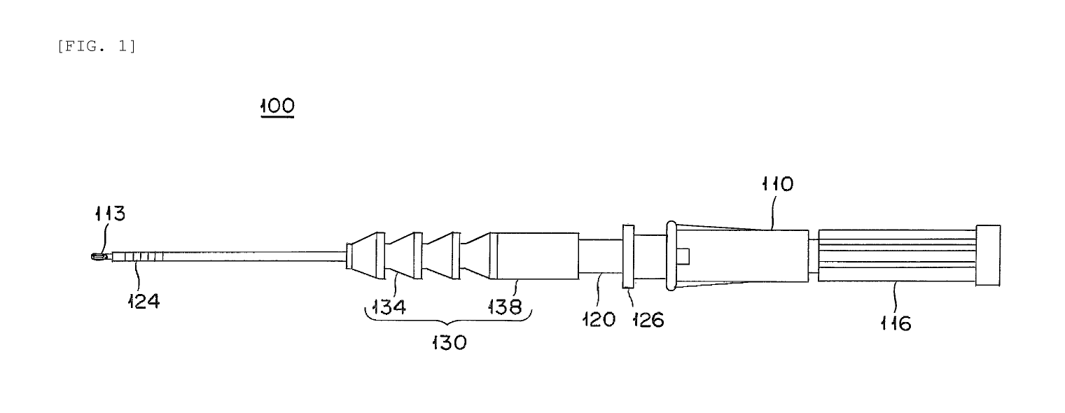

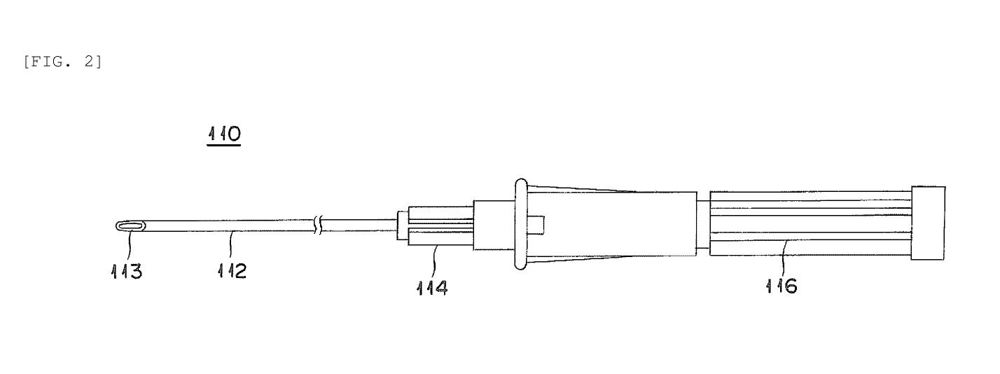

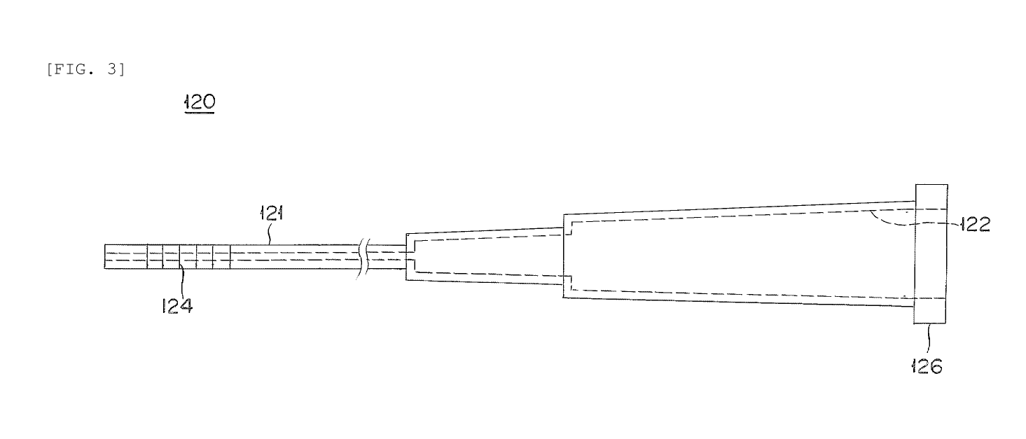

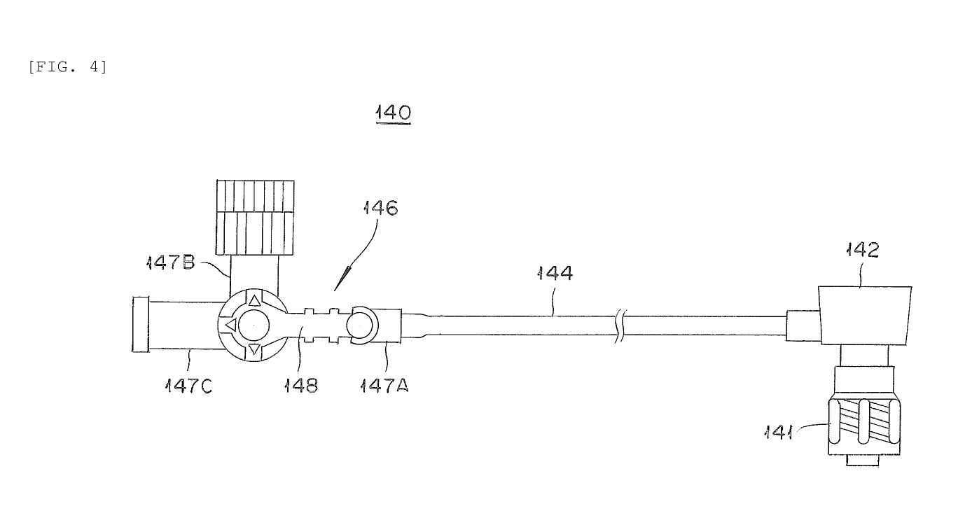

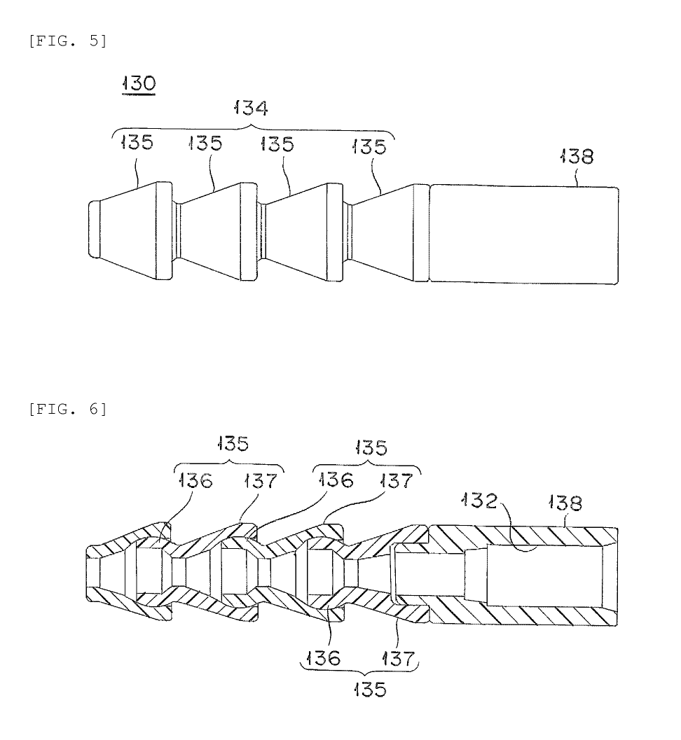

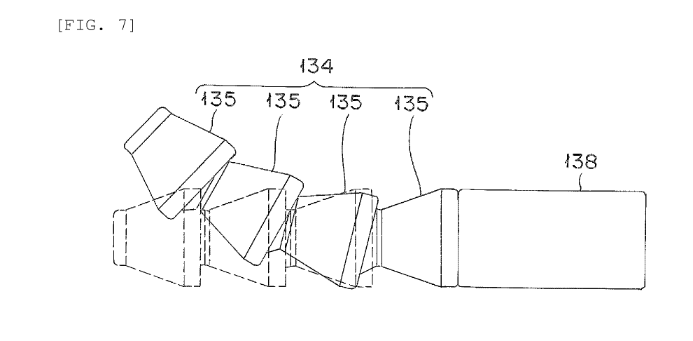

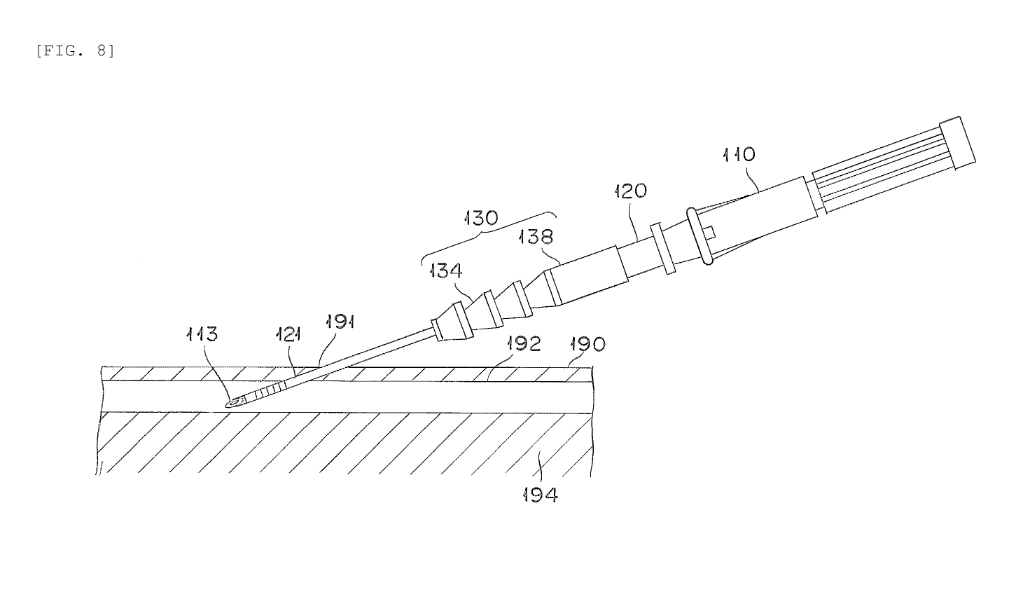

FIG. 1 is a side view illustrating a puncture device assembly according to Embodiment 1. FIG. 2 is a side view illustrating an inner needle shown in FIG. 1. FIG. 3 is a side view illustrating a sheath tube shown in FIG. 1. FIG. 4 is a side view illustrating a hemostasis valve attached to a connector shown in FIG. 1. FIGS. 5 and 6 are respectively a side view and a cross-sectional view illustrating a support portion shown in FIG. 1. FIG. 7 is a side view showing a curved state of the support portion shown in FIG. 1.

It is possible to directly insert a medical elongated object using a puncture device assembly 100 according to Embodiment 1. For example, the puncture device assembly is used for treating a lesion area generated in the blood vessels and has an introduction needle portion 110, a sheath tube 120, and a support portion 130. The length of the section introduced to the blood vessels in the puncture device assembly 100 is short and the diameter of the section is small. Moreover, the distance from the insertion point of the skin to the lesion area is short. Therefore, there is low possibility to damage the normal blood vessels and it is preferable in terms of less invasive treatment.

The medical elongated object is, for example, a balloon catheter. The insertion point on the skin can be areas such as the wrists, the arms, and thighs where blood vessels are generally secured, and are particularly the back of the knees, and the heels. The lesion area is, for example, a narrowed section or an occluded section of the blood vessels under the knees. Treatment is performed for reopening the blood vessels.

Next, the introduction needle portion 110, the sheath tube 120, and the support portion 130 will be sequentially described in detail.

As shown in FIG. 2, the introduction needle portion 110 has an inner needle 112, an inner needle hub 114, and a filter portion 116.

The inner needle 112 has a comparatively short length and is configured so as to be able to puncture the skin. Moreover, the inner needle is inserted to the sheath tube 120 such that a cutting edge 113 is exposed from the sheath tube 120. The constituent material of the inner needle 112 is metal or plastic. The metal is, for example, stainless steel, aluminium, aluminium alloy, titanium, and titanium alloy. The plastic is polyolefin or polyester. Examples of the polyolefin include high-density polyethylene and polypropylene and examples of the polyester include polyethylene terephthalate, polybutylene terephthalate, and polycyclohexane terephthalate.

The inner needle hub 114 is formed of a transparent material and is connected to the inner needle 112 which is installed thereto. The filter portion 116 has a ventilation filter which transmits gas but blocks the liquid. For example, when blood flows down through the inner needle 112 and reaches the ventilation filter, the ventilation is blocked due to the contact with the blood, and thus, it is possible to prevent the air from entering from the outside. In addition, it is possible to visually confirm that the inner needle 112 (cutting edge 113) has entered the blood vessel through the inflow of blood to the inner needle hub 114 and the filter portion 116.

As shown in FIG. 3, the sheath tube 120 has a lumen 122, a groove 124, and a connector 126. The sheath tube 120 also serves as an introducer sheath, that is, the sheath tube functions as the introducer sheath, and therefore, it is possible to simplify the procedure. Note that the reference numeral 121 indicates a distal portion of the sheath tube 120. It is preferable that the distal end of the sheath tube 120 have a tapered shape in order to percutaneously insert the needle into the body with low puncture resistance.

The lumen 122 has a valve body and is configured such that the inner needle 112 and the balloon catheter which is a medical elongated object can be inserted therethrough. The valve body has a Y-shaped slit or a cross-shaped or linear-shaped slit. The valve body is provided for securing liquid-tightness and prevents leakage of liquid such as blood.

The groove 124 is disposed at the distal portion 121 of the sheath tube 120 and is configured so as to generate a reflection echo with respect to ultrasonic waves. Accordingly, it is possible to specify the position of the distal portion 121 of the sheath tube 120 while visually confirming blood flow from an echo screen in real time even in a circumstance where it is difficult due to less blood flow to perform flashback through which the skin is punctured while confirming the blood flow, and therefore, the puncture device assembly is excellent in operability. The site having less blood flow is, for example, the peripheral blood vessels in the lower limbs.

The connector 126 has Luer taper, and is set to be able to, for example, attach and detach the introduction needle portion 110 and the hemostasis valve 140.

The main constituent material of the sheath tube 120 is at least one selected from a group consisting of polyolefine, polyamide, polyurethane, polyester, and fluorine-based resin. Examples of the polyolefin include high-density polyethylene and polypropylene; examples of the polyamide include nylon 6 and nylon 66; examples of the polyester include polyethylene terephthalate, polybutylene terephthalate, and polycyclohexane terephthalate; and examples of the fluorine-based resin include polytetrafluoroethylene and ethylene/tetrafluoroethylene-based copolymer.

In order to reduce friction resistance, it is preferable that hydrophilic treatment be performed on, the sheath tube 120, at least one portion of an outer surface. The hydrophilic treatment is physical activating treatment or coating treatment. Examples of the physical activating treatment include plasma treatment, glow discharge, corona discharge, and ultraviolet irradiation. Examples of the coating treatment include applying a surfactant, water soluble silicon, and a hydrophilic polymer material.

Examples of the hydrophilic polymer material include a single use or combined use of two or more of cellulose-based polymer material, polyethylene oxide-based polymer material, maleic anhydride polymer material, acrylamide-based polymer material, and a water-soluble nylon. Examples of the cellulose-based polymer material include hydroxypropyl-cellulose; examples of the polyethylene oxide-based polymer material include polyethylene glycol; examples of the maleic anhydride polymer material include methyl vinyl ether-maleic anhydride copolymer; and examples of the acrylamide-based polymer material include acrylamide-glycidyl methacrylate copolymer.

Examples of the constituent material of the valve body disposed in the lumen 122 of the sheath tube 120 include silicone rubber, latex rubber, butyl rubber, and isoprene rubber.

The hemostasis valve 140 is connected to a connector 126 of the sheath tube 120 and is used for preventing leakage of blood from the sheath tube 120. Moreover, as shown in FIG. 4, the hemostasis valve has a valve portion 142, a side tube 144, and a three-way stopcock 146.

The valve portion 142 is provided with a connector 141 having Luer taper and a lock mechanism, and can be attached to or detached from the connector 126, which has the Luer taper, of the sheath tube 120. The side tube 144 is connected to the valve portion 142 and the three-way stopcock 146.

The three-way stopcock 146 has a cock 148 for switching ports 147A to 147C and flow paths. The port 147A is connected to the side tube 144 and the ports 147B and 147C have Luer taper. The ports 147B and 147C are mounted with a syringe and are used for, for example, injecting liquid such as physiological saline or aspirating liquid flowing from the valve portion 142 (sheath tube 120). Note that the port 147B has a lock mechanism.

The hemostasis valve 140 is detachable. For this reason, it is possible to perform “aspiration” after ballooning that extends a narrowed blood vessel by expanding the blood vessel using a balloon, or after atherectomy that directly scraping cholesterol from the inside of the blood vessel using a catheter to which a cutter is attached therein, which is preferable. For example, after the ballooning or the atherectomy, it is possible to aspirate residues such as thrombus or cholesterol which flows by being accompanied by the blood flow without any interference during the ballooning or the atherectomy, by attaching the hemostasis valve 140 to which a suction syringe is connected. In addition, it is also possible to directly connect the syringe to the sheath tube 120 by removing the hemostasis valve 140. That is, it is possible to attach and detach the hemostasis valve 140 with respect to the connector 126 of the sheath tube 120 as necessary, and therefore, it is possible to apply the puncture assembly disclosed here to various procedures.

The support portion 130 is a protector which is used for preventing kink of the sheath tube 120 and is preferably formed of a hard plastic material. The support portion has a lumen 132 (refer to FIG. 6) which is configured such that the sheath tube 120 can be inserted in the lumen 132 and is disposed and mounted at the proximal portion of the sheath tube 120. The hard plastic material is, for example, polyamide such as nylon 6 or nylon 66.

The support portion 130 has a cylindrical body 134, which is bendable and can maintain a curved shape in a state where the sheath tube 120 is inserted, and a support portion base 138 to which the cylindrical body 134 is connected. The cylindrical body 134 is formed to have a plurality of cylindrical members 135 bendably connected to each other. The maximum bending angle is, for example, 45 degrees. The plurality of cylindrical members 135 are formed such that the adjacent members are bendably fitted to each other. With such a configuration, the cylindrical body is bendable and it is possible to maintain the curved shape.

More specifically, the cylindrical members 135 have a spherical distal portion 136 and a truncated cone-shaped base 137. The spherical distal portion 136 is rotatably fitted and contacted to the truncated cone-shaped base 137 of the adjacent cylindrical member 135. The curved shape of the cylindrical body 134 is maintained based on friction between the adjacent cylindrical members 135, that is, friction between the spherical distal portion 136 and the truncated cone-shaped base 137. In this manner, the configuration in which the cylindrical body is bendable and the curved shape can be maintained is achieved using a simple structure.

The bending angle can be controlled by adjusting the number of cylindrical members 135 to increase or decrease or by adjusting the movable range per cylindrical member 135 by changing the fitting structure between the truncated cone-shaped base 137 and the spherical distal portion 136. Note that the truncated cone-shaped base 137 of the cylindrical member 135 adjacent to the support portion base 138 is fixed to the support portion base 138. The cylindrical member 135 positioned at a distal end does not have the spherical distal portion 136. The support portion 130 can be bent in any directions.

Next, the method of using the puncture device assembly 100 will be described.

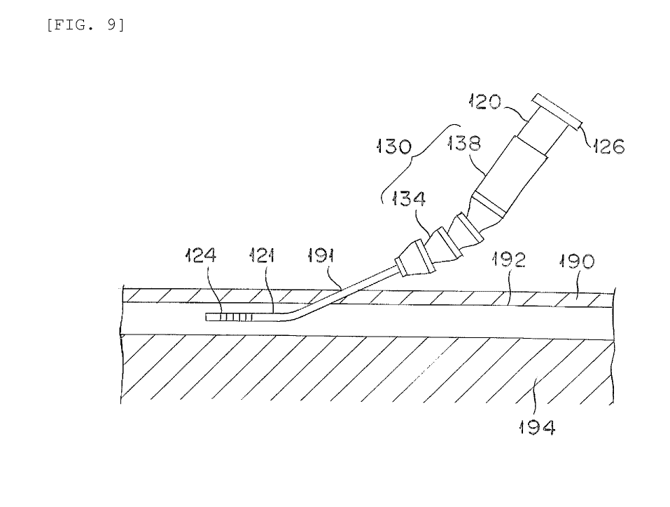

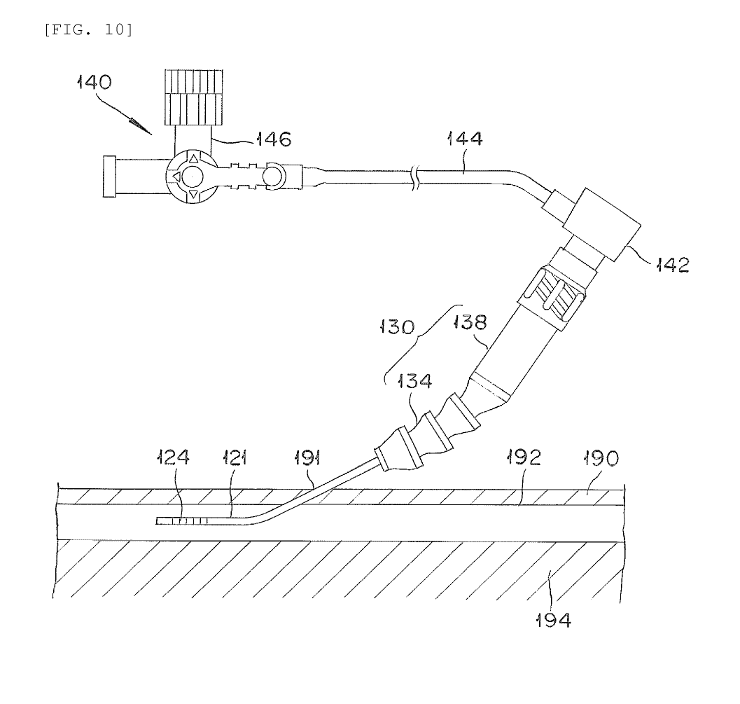

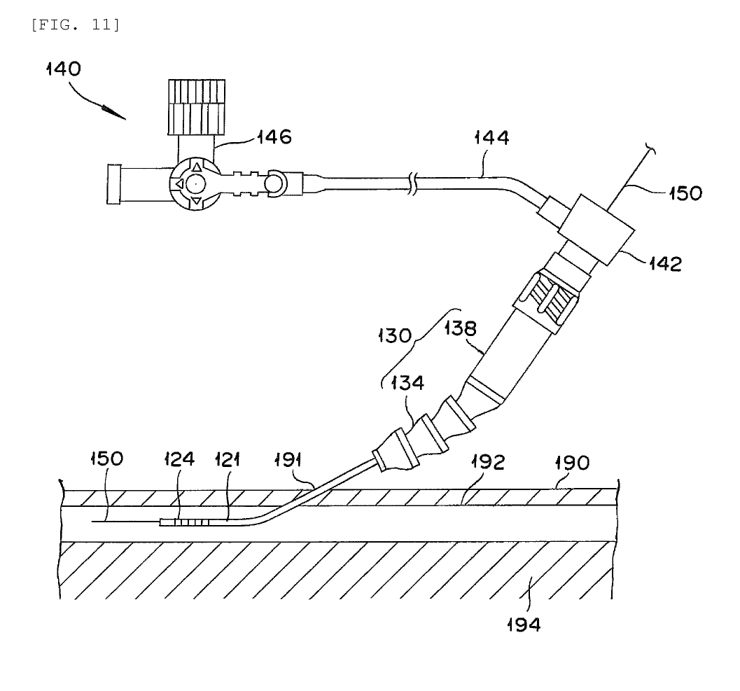

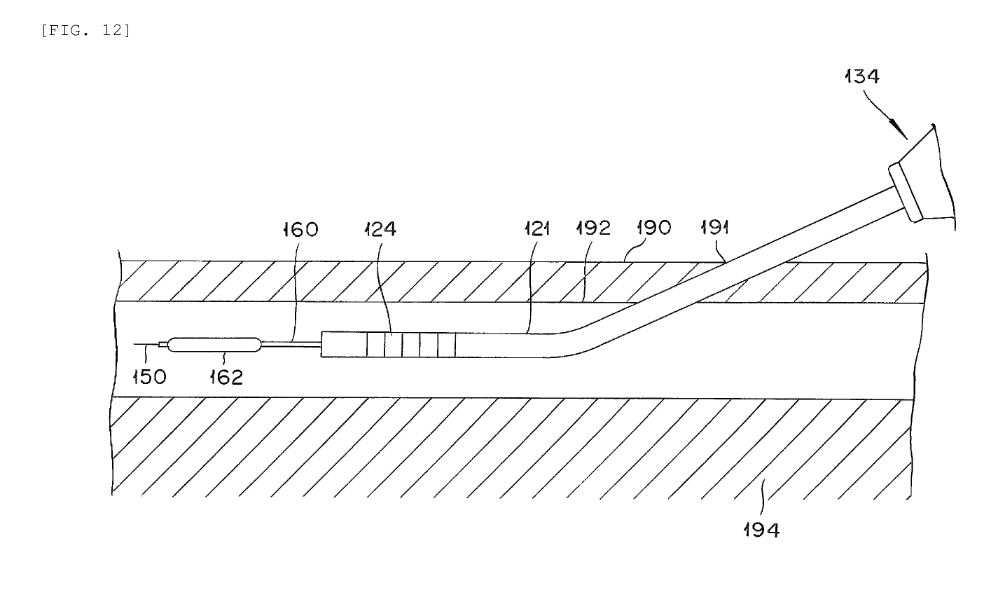

FIG. 8 is a side view illustrating puncturing. FIG. 9 is a side view illustrating removal of the inner needle. FIG. 10 is a side view illustrating mounting of the hemostasis valve. FIG. 11 is a side view illustrating insertion of a guide wire. FIG. 12 is a side view illustrating insertion of a balloon catheter.

First, small incision is made on the skin of an insertion point 191 using a surgical knife for cutting the skin as necessary. Then, as shown in FIG. 8, the skin 190 is punctured by the cutting edge 113 of the inner needle 112 which is exposed from the distal portion 121 of the sheath tube 120, and the distal portion 121 of the sheath tube 120 is introduced to the blood vessel 192 extending the inside of the body tissue 194. It is visually confirmed that the inner needle 112 (cutting edge 113) has entered the blood vessel through the inflow of blood to the inner needle hub 114 and the filter portion 116.

When the mounting of the introduction needle portion 110 is released by being removed from the sheath tube 120, the inner needle 112 is removed from the sheath tube 120. Then, as shown in FIG. 9, the distal portion 121 of the sheath tube 120 advances to be made coincident with the flow of the blood vessel 192, and the support portion 130 is bent to maintain its curved shape. At this time, the position of the distal portion 121 of the sheath tube 120 is managed by visually confirming the position of the groove 124 disposed at the distal portion 121 of the sheath tube 120 from an echo screen in real time.

As shown in FIG. 10, the connector 141 of the valve portion 142 of the hemostasis valve 140 is connected to the connector 126 of the sheath tube 120 and the hemostasis valve 140 is mounted to the sheath tube 120. That is, the sheath tube 120 functions as the introducer sheath, and therefore, the procedure is simplified. In addition, at this time, the curved shape of the support portion 130 is maintained, for example, fixed to 45 degrees, and lying down of the connector 126 which is a section positioned outside the sheath tube 120 is suppressed. Therefore, it is easy to mount the hemostasis valve 140. In addition, it is possible to omit the use of the sheath dilator, and therefore, it is possible to achieve less invasiveness with respect to the blood vessels.

As shown in FIG. 11, a guide wire 150 is inserted to the valve portion 142 of the hemostasis valve 140 starting from the flexible portion of the guide wire 150. The distal portion of the guide wire 150 passes through the lumen 122 of the sheath tube 120, protrudes from the distal portion 121 of the sheath tube 120, and advances toward a target section. The outer diameter of the guide wire 150 is, for example, 0.014 inch type. The length of the guide wire 150 inserted is appropriately adjusted by, for example, confirming a depth mark which is provided on the outer periphery of the guide wire 150. At this time, the curved shape of the support portion 130 is maintained, and therefore, it is easy to insert the guide wire 150.

A balloon catheter 160 is inserted to the valve portion 142 of the hemostasis valve 140, and as shown in FIG. 12, a balloon 162 disposed at the distal portion of the balloon catheter passes through the lumen 122 of the sheath tube 120, protrudes from the distal portion 121 of the sheath tube 120, advances along with the preceding guide wire 150, is disposed at a target section, and expands. For example, in a case where the target section is a narrowed section of the blood vessel, the balloon 162 reopens the narrowed blood vessel by extending the blood vessel. At this time, the curved shape of the support portion 130 is maintained, and therefore, it is easy to insert the balloon catheter 160.

It is possible to position the balloon 162 in the vicinity of the insertion point 191 of the sheath tube 120, that is, in the vicinity of the distal end of the sheath tube 120. This principle is dominant when, for example, the insertion point 191 is the back of the knees, and the heels, and the lesion area is positioned in the vicinity of the insertion point 191.

Next, Modification Examples 1 to 3 according to Embodiment 1 will be sequentially described.

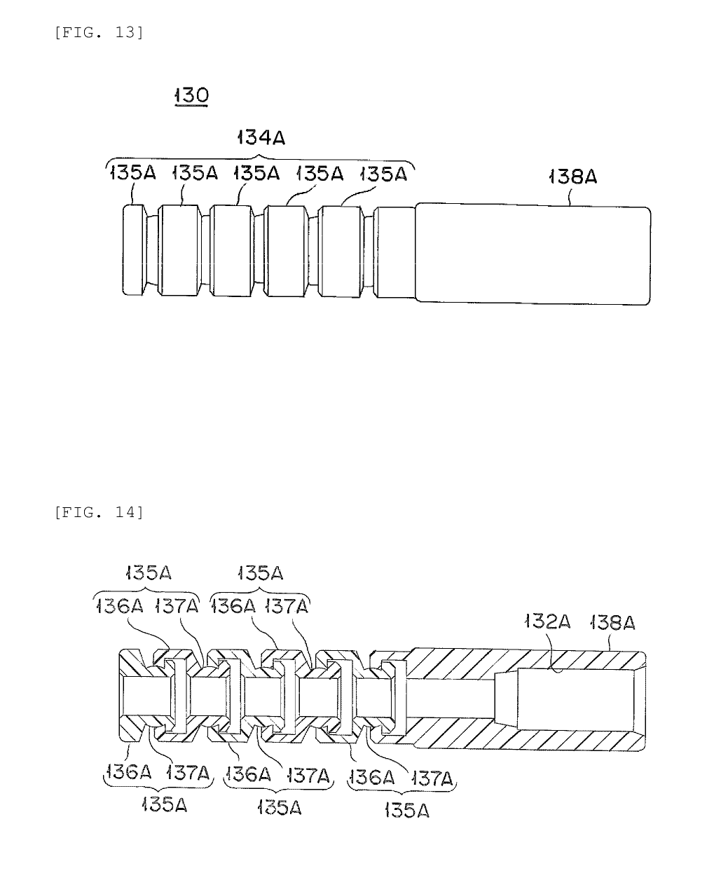

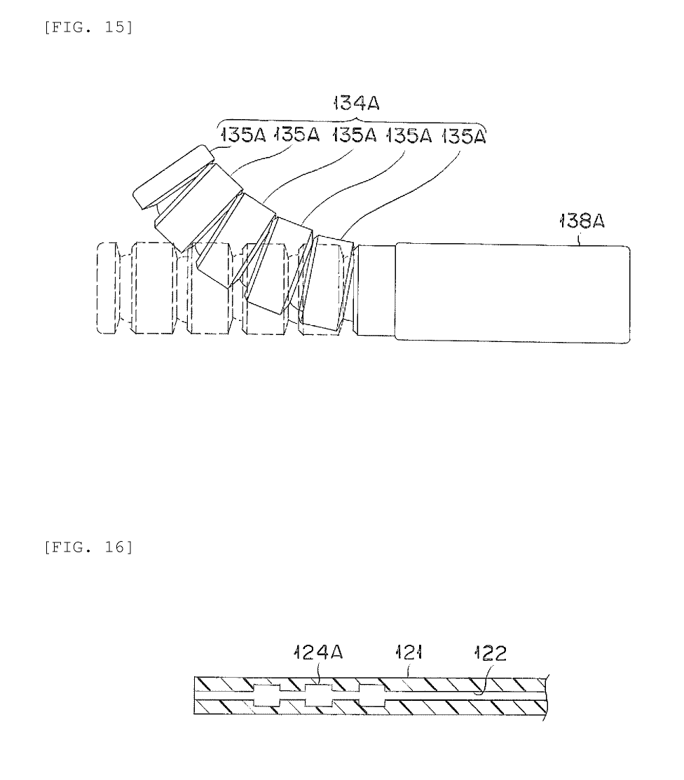

FIGS. 13 and 14 are side views illustrating Modification Example 1. FIG. 15 is a side view showing a curved state of Modification Example 1.

The cylindrical member 135 constituting the cylindrical body 134 of the support portion 130 is not limited to the mode of having the spherical distal portion 136 and the truncated cone-shaped base 137. For example, as shown in FIGS. 13 to 15, it is possible to apply a mode of having a diameter-enlarged distal portion 136A and a diameter-reduced base 137A and of showing a stepped shape.

In this case, the diameter-enlarged distal portion 136A is rotatably fitted and contacted to the diameter-reduced base 137A of the adjacent cylindrical member 135. The curved shape of the cylindrical body 134A is maintained based on friction between the adjacent cylindrical members, that is, friction between the diameter-enlarged distal portion 136A and the diameter-reduced base 137A.

The diameter-reduced base 137A of the cylindrical member 135A adjacent to the support portion base 138A is fixed to the support portion base 138A and the section to which the diameter-reduced base 137A is fixed in the support portion base 138A has a shape substantially coincident with the shape of the diameter-enlarged distal portion 136A. The diameter-enlarged distal portion 136A of the cylindrical member 135A positioned at the distal end does not have the fitting structure with the diameter-reduced base 137A.

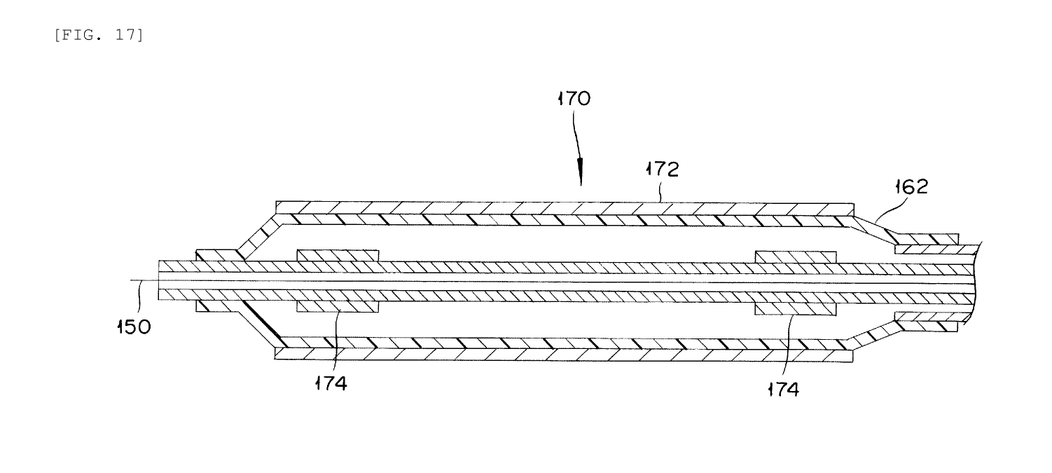

FIG. 16 is a cross-sectional view illustrating Modification Example 2.

It is preferable that the groove 124, which is disposed at the distal portion 121 of the sheath tube 120 be disposed on a surface of a lumen of the sheath tube 120. In this case, an air layer is interposed between the grooves 124 in a manner of being held by the grooves, and therefore, it is possible to efficiently generate the echo. The shape of the groove 124 and the number of the grooves disposed are not limited to the mode shown in FIG. 16. It is possible to appropriately apply machine (cutting) processing, laser processing, short blast processing, and the like for forming the groove 124.

FIG. 17 is a cross-sectional view illustrating Modification Example 3.

The medical elongated object which can be inserted to the lumen 122 of the sheath tube 120 is not limited to the balloon catheter. For example, it is possible to apply a stent delivery catheter 170. The stent delivery catheter 170 has a stent 172 disposed on the outer periphery of the balloon 162. The stent 172 is an in vivo indwelling object that holds the lumen by being contacted to and remaining in the inner surface of the narrowed section, and is configured to be expandable. In Modification Example 3, the balloon 162 is configured such that its diameter is enlarged by expanding the stent 172 disposed on the outer periphery of the balloon 162.

The reference numeral 174 shows a cylindrical marker. The marker 174 is formed of an X-ray impermeable material and it is possible to obtain a clear contrast image under X-ray illumination. Therefore, it is possible to easily confirm the positions of the balloon 162 and the stent 172. Examples of the X-ray impermeable materials include platinum, gold, tungsten, and iridium, or alloys thereof.

As described above, in Embodiment 1, the support portion has the cylindrical body which is bendable and can maintain a curved shape in a state where the sheath tube is inserted. Therefore, it is possible to suppress the lying down of the sheath tube or the kink of the sheath itself by maintaining the curved shape of the proximal portion of the sheath tube which is positioned in the vicinity of the insertion point. Accordingly, it is easy to insert the medical elongated object through the lumen of the sheath tube or to attach a medical instrument to the proximal portion of the sheath tube, for example. That is, it is possible to provide a puncture device assembly excellent in operability.

Next, Embodiment 2 will be described.

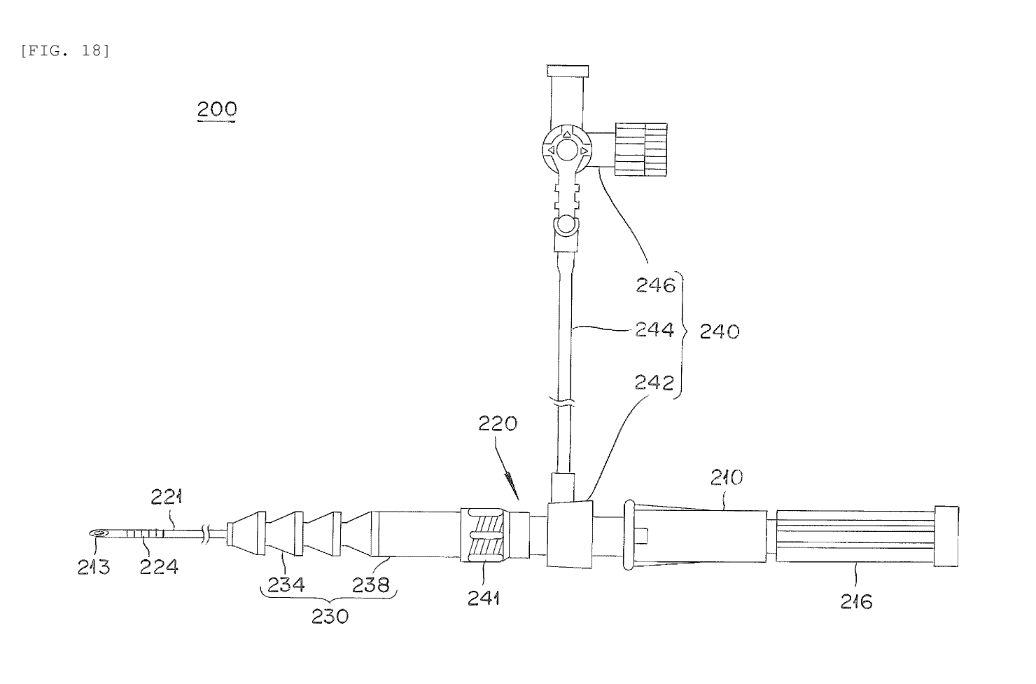

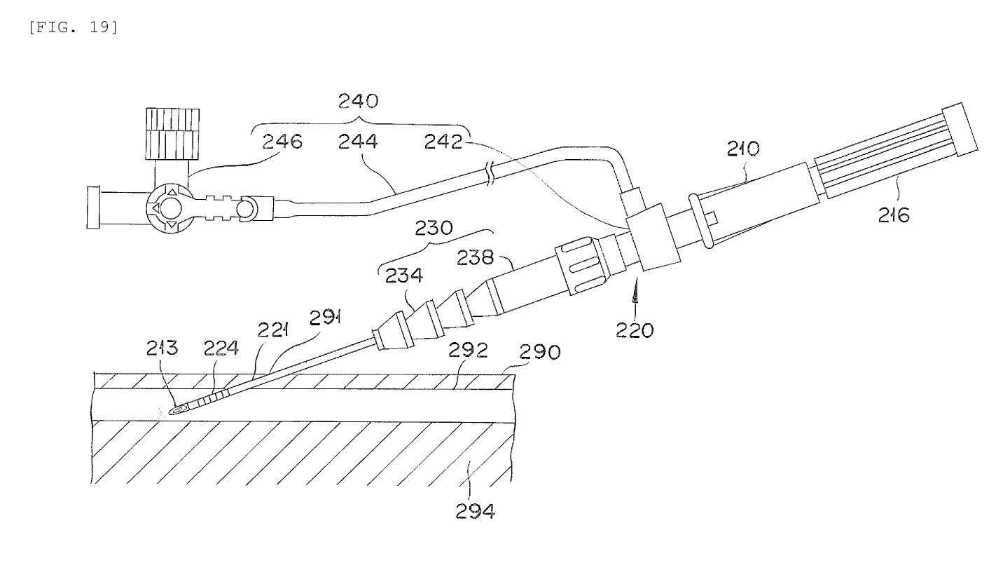

FIG. 18 is a side view illustrating a puncture device assembly according to Embodiment 2.

Embodiment 2 is generally different from Embodiment 1 with respect to the configuration of the sheath tube. Note that members having the same function as that of the Embodiment 1 are given of the similar reference numerals.

A puncture device assembly 200 according to Embodiment 2 has an introduction needle portion 210, a sheath tube 220, and a support portion 230. The introduction needle portion 210 has an inner needle, an inner needle hub, and a filter portion 216. The support portion 230 has a cylindrical body 234 which is bendable and can maintain a curved shape in a state where the sheath tube 220 is inserted, and a support portion base 238 to which the cylindrical body 234 is connected.

The sheath tube 220 has a lumen which is configured such that the inner needle and the medical elongated object can be inserted therethrough; a groove 224 which is disposed at a distal portion 221 of the sheath tube 220; and a hemostasis valve 240.

The hemostasis valve 240 has a valve portion 242, a side tube 244, and a three-way stopcock 246. The valve portion 242 has a connection portion 241 which is integrated with the sheath tube 220. The introduction needle portion 210 is inserted to be detachable. That is, the hemostasis valve 240 is integrated with the sheath tube 220, and therefore, the complexity in mounting operation of the hemostasis valve 240 is reduced.

The connection portion 241 is configured such that the introduction needle portion 210 which is inserted can be removed in a state where liquid-tightness is secured. The configuration of securing the liquid-tightness is not particularly limited, and for example, it is possible to use a valve body formed with an 0-ring or a cross cut.

Next, a method of using the puncture device assembly 200 will be described.

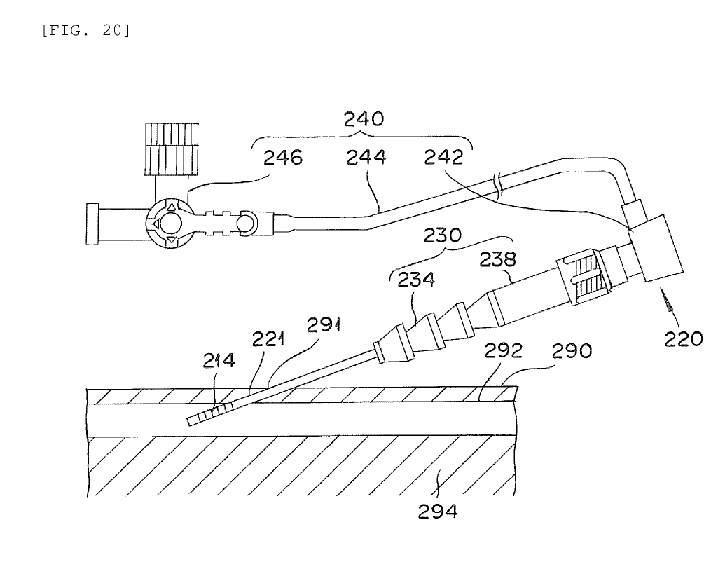

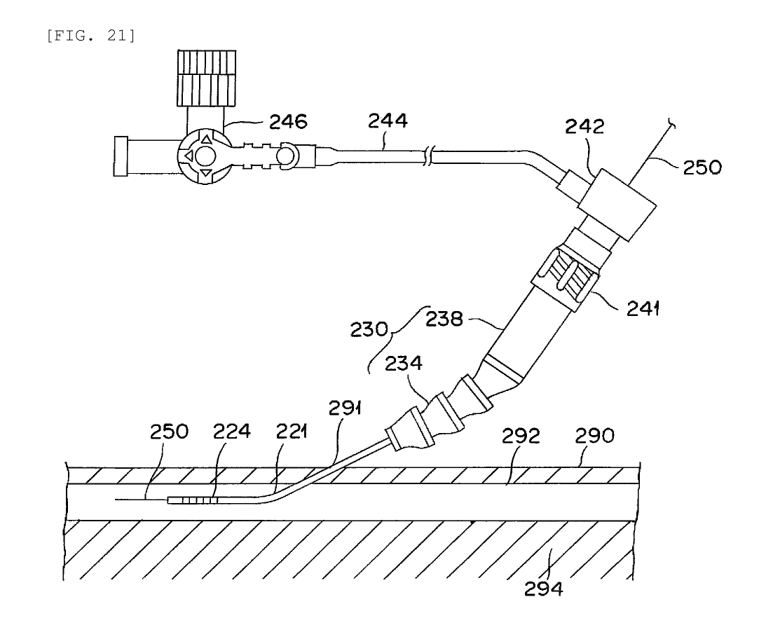

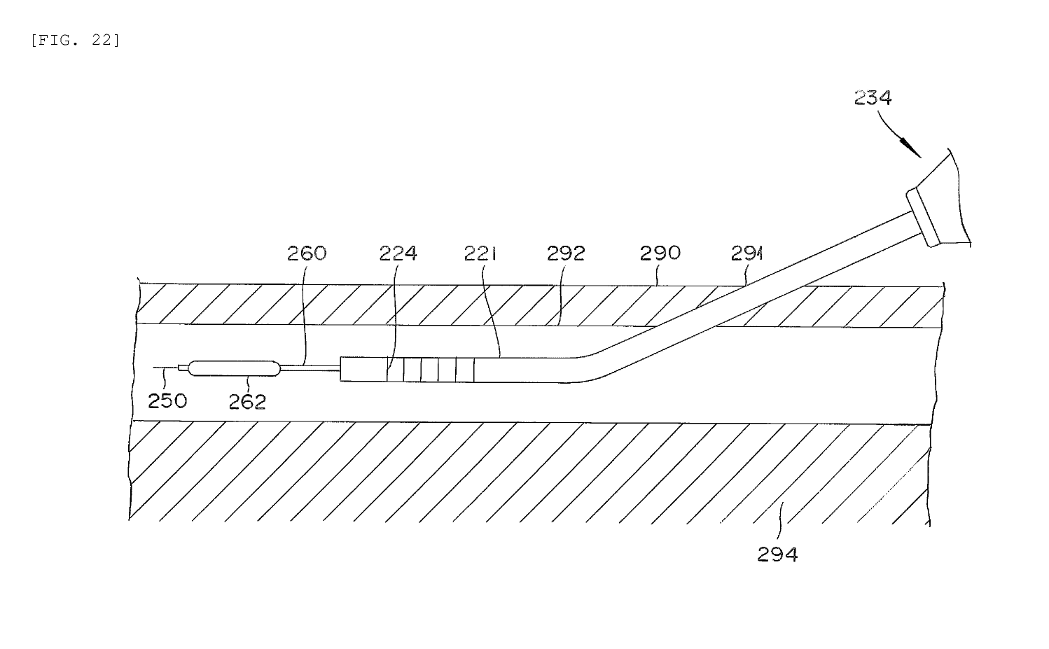

FIG. 19 is a side view illustrating puncturing, FIG. 20 is a side view illustrating removal of an inner needle, FIG. 21 is a side view illustrating insertion of a guide wire, and FIG. 22 is a side view illustrating insertion of a balloon catheter.

First, small incision is made on the skin of an insertion point 291 using a surgical knife for cutting the skin as necessary. Then, as shown in FIG. 19, the skin 290 is punctured by the cutting edge 213 of the inner needle 112 (refer to the reference numeral 112 of FIG. 2) which is exposed from the distal portion 221 of the sheath tube 220, and the distal portion 221 of the sheath tube 220 is introduced to the blood vessel 292 extending the inside of the body tissue 294. It is visually confirmed that the distal end of the inner needle has entered the blood vessel through the inflow of blood to the inner needle hub and the filter portion 216.

Moreover, as shown in FIG. 20, the mounting of the introduction needle portion 210 and the filter portion 216 is released by being removed from the sheath tube 220 and the inner needle is removed from the sheath tube 220.

Then, a guide wire 250 is inserted to the valve portion 242 of the hemostasis valve 240 starting from the flexible portion of the guide wire 250. The distal portion of the guide wire 250 passes through the inside of the introduction needle portion 210 positioned in the lumen of the sheath tube 220 integrated with the connection portion 241 of the valve portion 242, and advances until protruding from the distal portion 221 of the sheath tube 220. Then, as shown in FIG. 21, the distal portion 221 of the sheath tube 220 advances to be made coincident with the flow of the blood vessel 292 and the distal portion of the sheath tube 220 remains in a predetermined position. Subsequently, the support portion 230 is bent to maintain its curved shape. At this time, the position of the distal portion 221 of the sheath tube 220 is managed by visually confirming the position of the groove 224 which is disposed at the distal portion 221 of the sheath tube 220 through an echo screen in real time. The bent support portion 230 is fixed on the skin 290 by fixing means such as a tape.

A balloon catheter 260 is inserted to the valve portion 242 of the hemostasis valve 240, and as shown in FIG. 22, a balloon 262 disposed at the distal portion of the balloon catheter passes through the lumen (refer to the reference numeral 122 of FIG. 3) of the sheath tube 220, and protrudes from the distal portion 221 of the sheath tube 220. The balloon 262 advances along with the preceding guide wire 250, is disposed at a target section, and expands. At this time, the curved shape of the support portion 230 is maintained, and therefore, it is easy to insert the balloon catheter 260.

As described above, in Embodiment 2, the hemostasis valve is integrated with the sheath tube of the puncture device assembly, and therefore, the complexity in mounting operation of the hemostasis valve is reduced.

The detailed description above describes embodiments of a puncture assembly representing examples of the puncture assembly of the present invention. The invention is not limited, however, to the precise embodiments and variations described. Various changes, modifications and equivalents can effected by one skilled in the art without departing from the spirit and scope of the invention as defined in the accompanying claims. It is expressly intended that all such changes, modifications and equivalents which fall within the scope of the claims are embraced by the claims.

What is claimed is:

1. A puncture device assembly comprising:
an inner needle,
a sheath tube possessing a lumen through which the inner needle is configured to be inserted,
a support portion provided at a proximal portion of the sheath tube, the support portion being configured to prevent kink of the sheath tube; and
wherein the support portion includes a bendable cylindrical body configured to maintain a curved shape in a state where the sheath tube is inserted into a blood vessel.

2. The puncture device assembly according to claim 1,
wherein a medical elongated object is positionable in the lumen of the sheath tube, and
wherein the sheath tube is configured as an introducer sheath.

3. The puncture device assembly according to claim 2,
wherein the medical elongated object is at least one of a balloon catheter and a stent delivery catheter.

4. The puncture device assembly according to claim 1,
wherein the cylindrical body includes a plurality of cylindrical members bendably connected to each other and axially adjacent to each other, and
wherein the curved shape of the cylindrical body is maintained based on friction between each pair of axially adjacent cylindrical members.

5. The puncture device assembly according to claim 4,
wherein each of the cylindrical members has a spherical distal portion and a truncated cone-shaped base, and
wherein the spherical distal portion is rotatably fitted into the truncated cone-shaped base of the axially adjacent cylindrical members.

6. The puncture device assembly according to claim 4,
wherein each of the cylindrical members has a diameter-enlarged distal portion and a diameter-reduced base, and
wherein the diameter-enlarged distal portion is rotatably fitted to the diameter-reduced base of the axially adjacent cylindrical members.

7. The puncture device assembly according to claim 1, further comprising:
a hemostasis valve integrated with the sheath tube.

8. The puncture device assembly according to claim 7,
wherein a proximal portion of the sheath tube has a connector configured to attach and detach the hemostasis valve.

9. The puncture device assembly according to claim 8,
wherein the distal portion of the sheath tube has a groove that generates a reflection echo with respect to ultrasonic waves.

10. The puncture device assembly according to claim 9,
wherein the groove is disposed on a surface of the lumen of the sheath tube.

11. A method comprising:
puncturing a tissue with a cutting edge of a needle, the needle being positioned inside a lumen of a sheath tube wherein the cutting edge of the needle extends beyond a distal end portion of the sheath tube;
introducing the distal end portion of the sheath tube to a blood vessel extending inside the tissue;
removing the needle from the sheath tube;
bending a support portion of the sheath tube to be in a curved shape by applying an external force, the support portion of the sheath tube being provided proximal to the distal end portion of the sheath tube; and
wherein the support portion of the sheath tube maintains its curved shape when the external force is removed.

12. The method of claim 11,
wherein the support portion of the sheath tube includes a plurality of axially adjacent members, and
wherein the curved shape of the cylindrical body is maintained based on friction between each pair of axially adjacent cylindrical members.

13. The method according to claim 12,
wherein each of the axially adjacent members has a central axis, each of the central axis of the plurality of axially adjacent members being coaxial prior to the external force being applied;
wherein each of the central axis of the plurality of axially adjacent members are eccentric relative to an axially adjacent member when the external force is applied; and
wherein each of the central axis of the plurality of axially adjacent members remains eccentric relative to an axially adjacent member when the external force is subsequently removed.

14. The method according to claim 12,
wherein each of the axially adjacent members has a spherical distal portion and a truncated cone-shaped base; and
wherein the spherical distal portion is rotatably fitted into the truncated cone-shaped base of the axially adjacent cylindrical members.

15. The method according to claim 14,
wherein each of the cylindrical members has a diameter-enlarged distal portion and a diameter-reduced base, and
wherein the diameter-enlarged distal portion is rotatably fitted to the diameter-reduced base of the axially adjacent cylindrical members.

16. The method according to claim 11, further comprising:
inserting a medical elongated object in the sheath tube, wherein the sheath tube is configured as an introducer sheath.

17. The method according to claim 16,
wherein the medical elongated object is at least one of a balloon catheter and a stent delivery catheter.
